(19) United States
(12) Patent Application Publication
(10) Pub. No.: US 2024/0051856 A1
(43) Pub. Date: Feb. 15, 2024

(54) BIOFILM GROWTH-CONTROLLING DEVICE AND METHOD

(71) Applicants: EWHA UNIVERSITY - INDUSTRY COLLABORATION FOUNDATION, Seoul (KR); KOREA UNIVERSITY RESEARCH AND BUSINESS FOUNDATION, Seoul (KR)

(72) Inventors: Ahjeong Son, Seoul (KR); Jeongeun Lee, Chungcheongbuk-do (KR); Beelee Chua, Seoul (KR); Donghyun Lee, Seoul (KR)

(21) Appl. No.: 18/446,773

(22) Filed: Aug. 9, 2023

(30) Foreign Application Priority Data
Aug. 9, 2022 (KR) 10-2022-0099344

(51) Int. Cl. C02F 3/00

(52) U.S. Cl. CPC C02F 3/005; C02F 3/006; C02F 2303/20; C02F 2101/30

(57) ABSTRACT

Provided is an electrode structure including a first electrode pattern including a plurality of electrode fingers extending in one direction; and a second electrode pattern which is provided between the plurality of electrode fingers provided in the first electrode pattern to form an interdigitated pattern with the first electrode pattern, wherein a fringe field is generated between the first electrode pattern and the second electrode pattern to regulate growth of a biofilm which is provided on the surface of at least one of the first electrode pattern and the second electrode pattern.

### RELATED APPLICATIONS

This application claims priority to Korean Patent Application No. 10-2022-0099344, entitled BIOFILM GROWTH-CONTROLLING DEVICE AND METHOD, filed Aug. 9, 2022, incorporated by reference herein.

### BACKGROUND OF THE INVENTION

### 1. Field of the Invention

The present disclosure relates to a biofilm-regulating device and a method of regulating a biofilm.

### 2. Description of the Related Art

As a consortium of bacterial cells protected by a hydrogel-like polymeric matrix, a biofilm can thrive in a vast myriad of hostile environments ranging from the inhospitable abyss of ocean trenches to the intimate lining of the human gut. Biofilms are generally treated as objects to be removed, but there is room for industrial application thereof. For example, in a wastewater treatment process, biofilms are used in systems such as rotating biological contactors, trickling filters, and fluidized bed and membrane bioreactors. Thriving biofilms on surfaces can efficiently remove nutrients and organic pollutants such as nitrogen and phosphorous from the raw water. However, their excessive growth would also incapacitate the wastewater treatment process due to biofouling.

Therefore, the industrial application of biofilms requires a delicate balance between promoting and inhibiting biofilm growth, which can be achieved through periodic maintenance (e.g., backwashing, air scouring, or parts replacement). However, such maintenance causes problems in that the process must be temporarily stopped and costs are continuously incurred. Accordingly, there is a need for a new method capable of regulating biofilm growth in the process without stopping the process for maintenance.

An object of the present disclosure is to provide an electrode structure capable of regulating biofilm growth even at low voltage and a water treatment device capable of treating contaminated water by including the electrode structure.

### SUMMARY OF THE INVENTION

According to one embodiment of the present disclosure, there is provided an electrode structure including a first electrode pattern including a plurality of electrode fingers extending in one direction; and a second electrode pattern which is provided between the plurality of electrode fingers provided in the first electrode pattern to form an interdigitated pattern with the first electrode pattern, wherein a fringe field is generated between the first electrode pattern and the second electrode pattern to regulate growth of a biofilm which is provided on the surface of at least one of the first electrode pattern and the second electrode pattern.

According to one embodiment of the present disclosure, there is provided the electrode structure, wherein both the first electrode pattern and the second electrode pattern are covered with a thin electrical insulating layer, wherein the first electrode pattern is connected with electrical power, and the second electrode pattern is grounded.

According to one embodiment of the present disclosure, there is provided the electrode structure, wherein oscillating electrical signals are applied to the first electrode pattern.

According to one embodiment of the present disclosure, there is provided the electrode structure, wherein the first electrode pattern and the second electrode pattern are provided on the same side of a support.

According to one embodiment of the present disclosure, there is provided the electrode structure, further including a third electrode pattern and a fourth electrode pattern which are provided on the side other than the side of the support on which the first electrode pattern and the second electrode pattern are provided, wherein the third electrode pattern includes a plurality of electrode fingers extending in one direction, and the fourth electrode pattern is provided between the plurality of electrode fingers provided in the third electrode pattern to form an interdigitated pattern with the third electrode pattern, wherein a fringe field is generated between the third electrode pattern and the fourth electrode pattern.

According to one embodiment of the present disclosure, there is provided the electrode structure, wherein the surface of each of the first electrode pattern and the second electrode pattern is covered with an insulating layer.

According to one embodiment of the present disclosure, there is provided a water treatment device including a first electrode pattern including a plurality of electrode fingers extending in one direction; a second electrode pattern which is provided between the plurality of electrode fingers provided in the first electrode pattern to form an interdigitated pattern with the first electrode pattern; and a biofilm which is provided on the surface of at least one of the first electrode pattern and the second electrode pattern, wherein both the first electrode pattern and the second electrode pattern are covered with a thin electrical insulating layer, wherein the biofilm is provided in the flow path of raw water or in a chamber into which raw water is introduced so that the biofilm may be brought into contact with raw water to be treated, thereby removing organic pollutants in raw water, and a fringe field is generated between the first electrode pattern and the second electrode pattern to regulate growth of the biofilm.

According to one embodiment of the present disclosure, there is provided the water treatment device, wherein the first electrode pattern and the second electrode pattern are covered with an insulating layer, respectively, and the biofilm is provided on the insulating layer.

According to one embodiment of the present disclosure, there is provided the water treatment device, further including an electrical power controlling unit for generating a fringe field by applying electrical power to the first electrode pattern when the thickness of the biofilm is examined and is greater than a predetermined value.

According to one embodiment of the present disclosure, there is provided the water treatment device, wherein the distance between the electrode fingers provided in the first electrode pattern and the electrode fingers provided in the second electrode pattern may be adjusted according to the thickness of the biofilm and the throughput of the raw water.

According to one embodiment of the present disclosure, there is provided a water treatment method, the method including the steps of growing a biofilm on an electrode structure; bringing the electrode structure, to which the biofilm is attached, into contact with raw water to be treated; and generating a fringe field from the electrode structure by examining the growth state of the biofilm.

According to one embodiment of the present disclosure, there is provided the water treatment method, wherein the electrode structure includes a first electrode pattern including a plurality of electrode fingers extending in one direction; a second electrode pattern which is provided between the plurality of electrode fingers provided in the first electrode pattern to form an interdigitated pattern with the first electrode pattern; and the fringe field is generated between the first electrode pattern and the second electrode pattern to regulate growth of the biofilm which is provided on the surface of at least one of the first electrode pattern and the second electrode pattern.

### BRIEF DESCRIPTION OF THE DRAWINGS

FIG. 1 shows a perspective view of an electrode structure according to one exemplary embodiment of the present disclosure;

FIG. 2 shows a plan view of the top and bottom sides of the electrode structure according to one exemplary embodiment of the present disclosure;

FIG. 3 shows a cross-sectional view of the electrode structure according to one exemplary embodiment of the present disclosure;

FIG. 4 shows a cross-sectional view of a water treatment device including the electrode structure according to one exemplary embodiment of the present disclosure;

FIG. 5 shows cross-sectional views of water treatment devices according to the prior art;

FIG. 6 shows a cross-sectional view of the water treatment device according to one exemplary embodiment of the present disclosure;

FIG. 7 shows a flowchart illustrating a water treatment method according to one exemplary embodiment of the present disclosure;

FIG. 8A to 8H shows a method of preparing the electrode structure according to one exemplary embodiment of the present disclosure;

FIG. 9A to 9C shows results of analyzing a fringe field generated by the electrode structure; and

FIG. 10A to 10E are graphs showing results of measuring optical density of a biofilm fabricated according to one exemplary embodiment of the present disclosure.

### DETAILED DESCRIPTION OF THE PREFERRED EMBODIMENTS

While the present disclosure may be variously modified and have various forms, specific exemplary embodiments will be illustrated in drawings and explained in detail in this description. It should be understood, however, that the description is not intended to limit the present disclosure to the particular forms disclosed, but on the contrary, the intention is to cover all modifications, equivalents, and alternatives falling within the spirit and scope of the disclosure.

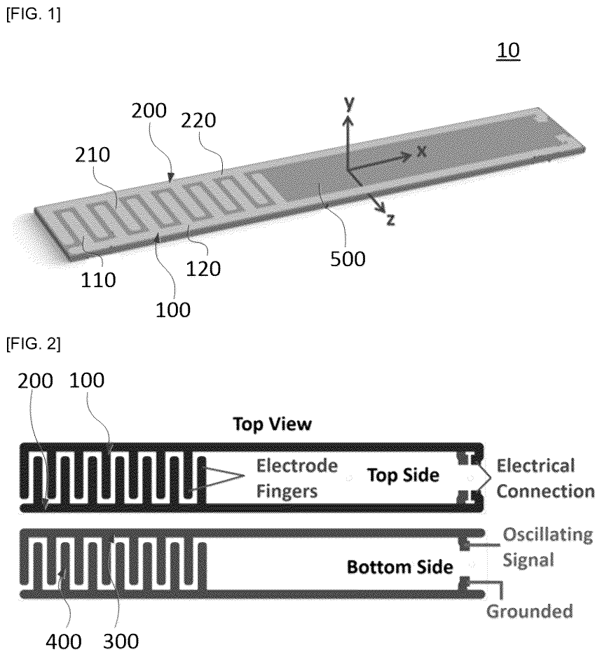

FIG. 1 shows a perspective view of an electrode structure according to one exemplary embodiment of the present disclosure.

Referring to FIG. 1, there is provided an electrode structure 10 including a first electrode pattern 100 including a plurality of electrode fingers 110 extending in one direction; and a second electrode pattern 200 which is provided between the plurality of electrode fingers 110 provided in the first electrode pattern 100 to form an interdigitated pattern with the first electrode pattern 100, wherein a fringe field is generated between the first electrode pattern 100 and the second electrode pattern 200 to regulate growth of a biofilm which is provided on the surface of at least one of the first electrode pattern 100 and the second electrode pattern 200.

As described above, the electrode structure 10 has the interdigitated structure of the first electrode pattern 100 and the second electrode pattern 200 by providing the plurality of electrode fingers 110, 210, and therefore, the fringe field is generated on the surfaces of the first electrode pattern 100 and the second electrode pattern 200. The fringe field is generated such that it diverges from the surfaces of the first electrode pattern 100 and the second electrode pattern 200 and then converges to the surfaces thereof again. In the electrode structure 10 according to one embodiment of the present disclosure, a diverging-converging electric field is generated, as described above, on the surfaces of the first electrode pattern 100 and the second electrode pattern 200, and configuration of the electrode structure 10 may be freely arranged, and its application is excellent, unlike in the prior art, wherein the electric field is generated only between two facing electrodes.

The configuration of the electrode structure 10 may be determined according to the shape of the first electrode pattern 100 and the second electrode pattern 200. However, since the first electrode pattern 100 and the second electrode pattern 200 are provided, in which the plurality of electrode fingers 110 and 210 are interdigitated, the electrode structure 10, which is an assembly thereof, has a configuration extending in one direction. In this regard, along the direction in which the electrode structure 10 extends, the plurality of electrode fingers 110 and 210 are alternately provided and may be provided in the interdigitated pattern. The electrode structure 10 may have various shapes such as a rectangle, an ellipse, a trapezoid, a triangle, etc.

On the electrode structure 10, the first electrode pattern 100 and the second electrode pattern 200 are provided in the interdigitated pattern.

The first electrode pattern 100 includes the plurality of electrode fingers 110 extending in one direction. In this regard, the extending direction of the electrode fingers 110 may be different from the extending direction of the electrode structure 10 described above. For example, as shown in the drawing, the electrode fingers 110 may extend in the z direction, and the electrode structure 10 may extend in the x direction.

The plurality of electrode fingers 110 provided in the first electrode pattern 100 may be provided on the same plane. Specifically, on the xz plane in the drawing, the plurality of electrode fingers 110 may be provided by spacing apart at predetermined intervals.

The first electrode pattern 100 may be made of a conductive material. The first electrode pattern 100 may be formed of, for example, a metal having excellent electrical conductivity or an inorganic compound such as graphene or indium tin oxide (ITO). Considering that the electrode structure 10 including the first electrode pattern 100 may be used in water, the first electrode pattern 100 may be provided using a thin overlay of electrically insulating material such as epoxy to prevent direct contact with water. The electrical insulating layer provided on the first electrode pattern 100 (and/or the second electrode pattern 200) has thickness of 0.1 μm to 200 μm. The thickness of the electrical insulating layer can be decided based on the form or material of electrode pattern.

A method of providing the first electrode pattern 100 is not particularly limited. For example, the first electrode pattern 100 may be provided in various forms such as screen-printing, lithography, lift-off, electroplating, 3D printing etc.

The number of the electrode fingers 110 provided in the first electrode pattern 100 may be two or more. By providing the plurality of electrode fingers 110, a fringe field may be provided over a wide area on the surface of the electrode structure 10. Since the generated fringe field may regulate growth of the biofilm provided on the surface of the electrode structure 10, it is possible to provide the biofilm over a wide area and to regulate growth thereof.

The electrode fingers 110 provided in the first electrode pattern 100 may be connected to a first electrode pattern wiring unit 120 connected to electrical power in order to supply electrical power to the electrode fingers 110. The first electrode pattern wiring unit 120 is connected to an external electrical power and may extend along the extending direction of the electrode structure 10. In some cases, the electrode fingers 110 and the first electrode pattern wiring unit 120 may be provided on different planes.

An oscillating electrical signal may be applied to the first electrode pattern 100 through the first electrode pattern wiring unit 120. Specifically, alternating current electrical power of sinusoidal wave may be connected to the first electrode pattern 100 to apply the oscillating electrical signal, and the oscillating electrical signal may generate a fringe field.

Next, the second electrode pattern 200 is provided in an interdigitated pattern with the first electrode pattern 100 described above.

Like the first electrode pattern 100, the second electrode pattern 200 includes a plurality of electrode fingers 210. At this time, the electrode fingers 210 included in the second electrode pattern 200 may extend in one direction. In this regard, the extending direction of the electrode fingers 210 included in the second electrode pattern 200 may be opposite to the extending direction of the electrode fingers 110 included in the first electrode pattern 100. As the two electrode fingers 110 and 210 extend in opposite directions, they may be provided in the interdigitated structure.

The second electrode pattern 200 does not necessarily exist on the same plane as the first electrode pattern 100. In the drawing, both the second electrode pattern 200 and the first electrode pattern 100 are illustrated to exist on the xz plane, if necessary, the two members may be provided on different planes. Even in this case, the second electrode pattern 200 and the first electrode pattern 100 are provided in the interdigitated structure, and the electrode fingers 110 and 210 may be obliquely interdigitated with each other to form an acute or obtuse angle.

A plurality of electrode fingers 210 may also be provided in the second electrode pattern 200. In this regard, the number of the electrode fingers 210 provided in the second electrode pattern 200 may be the same as the number of the electrode fingers 110 provided in the first electrode pattern 100. Accordingly, the electrode fingers 210 provided in the second electrode pattern 200 and the electrode fingers 110 provided in the first electrode pattern 100 may be matched 1:1, and the fringe field may be generated between the two electrode fingers 110 and 210 which are matched 1:1.

The electrode fingers 210 provided in the second electrode pattern 200 may be connected to a second electrode pattern wiring unit 220. The second electrode pattern wiring unit 220 may be grounded. Accordingly, electrical power may be supplied only to the first electrode pattern 100 and the fringe field may be generated between the grounded second electrode pattern 200 and the first electrode pattern 100.

The second electrode pattern 200 may also be made of a conductive material. The second electrode pattern 200 may be formed of, for example, a metal having excellent electrical conductivity or an inorganic compound graphene such as indium tin oxide (ITO). The second electrode pattern 200 may be provided with a material which is the same as or different from the first electrode pattern 100. When the second electrode pattern 200 and the first electrode pattern 100 are provided with the same material, they may be provided at the same time.

The strength of the fringe field may be adjusted by adjusting the gap between the electrode fingers 110 and 210 included in the second electrode pattern 200 and the first electrode pattern 100 and the width of respective electrode fingers 110 and 210. Thus, the strength of the fringe field may be adjusted without changing other parameters such as voltage. Accordingly, the device may be configured such that the gap between the electrode fingers 110 and 210 in the electrode structure 10 is variable. In this case, the density and extent of the fringe field may be adjusted without changing the fundamental design of the electrode structure. Specifically, the strength of the fringe field may be enhanced by narrowing the width and gap of the electrode fingers 110 and 210.

The second electrode pattern 200 and the first electrode pattern 100 may be provided on a support 500. The support 500 supports the first electrode pattern 100 and the second electrode pattern 200.

The support 500 is non-conductive and may be provided with a material with excellent mechanical and chemical stability. For example, the support 500 may be provided to include silicone, fiberglass, or the like. The shape of the support 500 may be determined by considering the shapes of the first electrode pattern 100 and the second electrode pattern 200 so as to support the first electrode pattern 100 and the second electrode pattern 200. In addition, the support 500 may be provided with flexibility, as needed.

As described above, the support 500 supports the first electrode pattern 100 and the second electrode pattern 200, wherein the first electrode pattern 100 and the second electrode pattern 200 may be provided on one side of the support 500. The first electrode pattern 100 and the second electrode pattern 200 may be provided on the same side of the support 500. However, in some cases, the first electrode pattern 100 and the second electrode pattern 200 may be provided on different sides of the support 500, and at least a part of the first electrode pattern 100 and the second electrode pattern 200 may be provided on different sides of the support 500. For example, the first electrode pattern wiring unit 120 included in the first electrode pattern 100 and the second electrode pattern wiring unit 220 included in the second electrode pattern 200 are provided on the side of the support 500, and the electrode fingers 110 and 210 connected to any one thereof may be provided on the top side of the support 500.

The support 500 may have a flat surface, in some cases, it may have a non-flat surface, that is, a surface including curves or irregularities. The first electrode pattern 100 and the second electrode pattern 200 may be made to conform over curved topologies. The first electrode pattern 100 and the second electrode pattern 200 may be easily screen-printed on flexible materials such as polyamide and polyester. Therefore, since the electrode structure 10 may be configured by providing the first electrode pattern 100 and the second electrode pattern 200 for the support 500 of various shapes and materials, the electrode structure 10 is very scalable.

The electrode structure 10 including the above-described members regulates growth of the biofilm provided on the surface of the electrode structure 10 by using a fringe field generated on the surface. Here, the biofilm is a consortium of bacterial cells, and refers to microbial growth in the form of a layer on the surface of the electrode structure 10.

Traditionally, an ultraviolet irradiation method has been performed to inhibit biofilm growth. However, in a turbid solution, UV rays are blocked by suspended solids and may not be effectively irradiated onto the biofilm. Another method is a method of inhibiting biofilm growth via the addition of specific pharmaceutical means. However, these methods are also ill-suited because they present unknown collaterals such as anti-microbial resistance and unwanted mutations. Further, the above-describe method has a limitation in that pharmaceuticals themselves are also one of the key pollutants to be removed from the water. Other possible approaches to reduce biofilm growth include ozonation, chlorination, and sonication. However, ozonation and chlorination are also unsuitable because they will result in corrosion of metal alloy surfaces as well as production of detrimental byproducts. Sonication is not scalable in terms of electrical power (400 W for a 200 mL sample).

According to the present disclosure, electric field in the form of fringe field is used to inhibit biofilm growth. Pulsed electric field (several kV/cm) creates pores in the phospholipid bilayers (cell membrane). Electric field is suitable for use in inhibiting biofilm growth in terms of its simplicity, efficacy, and side effects.

The electrode structure described above may be implemented in a form different from that shown in the drawings. For example, additional electrode patterns, in addition to the first electrode pattern and the second electrode pattern, may be provided in order to widen the area where the fringe field is generated.

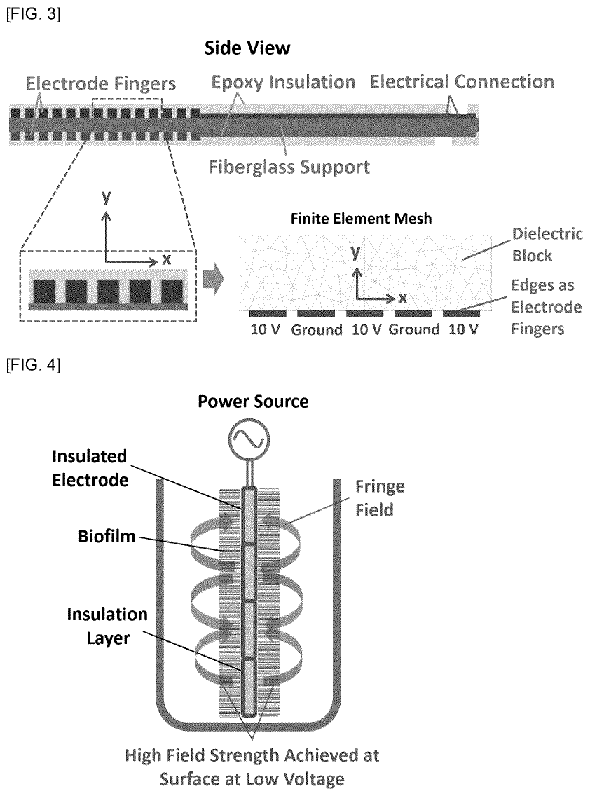

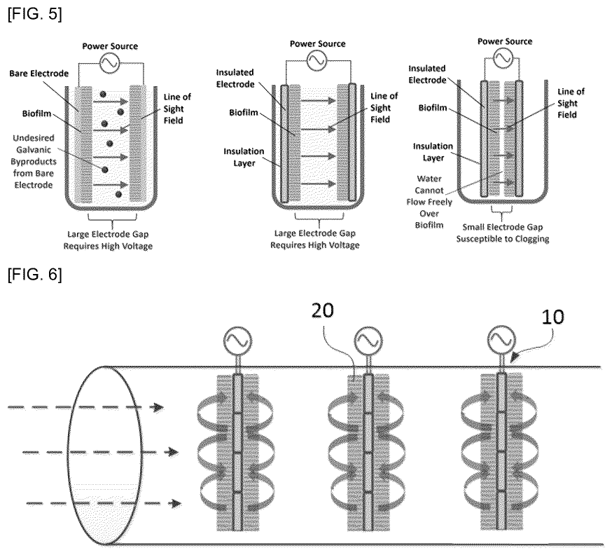

FIG. 2 shows a plan view of the top and bottom sides of the electrode structure according to one exemplary embodiment of the present disclosure. FIG. 3 shows a cross-sectional view of the electrode structure according to one exemplary embodiment of the present disclosure.

Referring to FIGS. 2 and 3, the electrode structure may further include a third electrode pattern 300 and a fourth electrode pattern 400, in addition to the first electrode pattern 100 and the second electrode pattern 200.

In this regard, the first electrode pattern 100 and the second electrode pattern 200 may be provided on the same side of the support, and the third electrode pattern 300 and the fourth electrode pattern 400 are provided on a side different from the side on which the first electrode pattern 100 and the second electrode pattern 200 are provided. For example, the first electrode pattern 100 and the second electrode pattern 200 may be provided on the top side of the support, and the third electrode pattern 300 and the fourth electrode pattern 400 may be provided on the bottom side of the support.

The third electrode pattern 300 may have a pattern corresponding to the first electrode pattern 100, and the fourth electrode pattern 400 may have a pattern corresponding to the second electrode pattern 200. Further, the third electrode pattern 300 and the first electrode pattern 100 may be electrically connected, and the fourth electrode pattern 400 and the second electrode pattern 200 may also be electrically connected. Therefore, as described above, when the first electrode pattern 100 is connected to electrical power and the second electrode pattern 200 is grounded, electrical power is also supplied to the third electrode pattern 300 which is connected to the first electrode pattern 100, and the fourth electrode pattern 400 which is connected to the second electrode pattern 200 may be grounded. Accordingly, as a fringe field is generated between the first electrode pattern 100 and the second electrode pattern 200, a fringe field may also be generated between the third electrode pattern 300 and the fourth electrode pattern 400. In the above case, since the first electrode pattern 100 and the third electrode pattern 300 are electrically connected and electrical power of the same waveform and frequency is applied, the two electrode patterns may function as one connected conductor. Accordingly, the fringe field generated between the first electrode pattern 100 and the second electrode pattern 200 may be the same as the fringe field generated between the third electrode pattern 300 and the fourth electrode pattern 400.

As needed, the third electrode pattern 300 and the fourth electrode pattern 400 may not be connected to the first electrode pattern 100 and the second electrode pattern 200, respectively. In other word, the first electrode pattern is electrically separated from the third electrode pattern and the second electrode pattern is electrically separated the fourth electrode pattern. In this case, separate electrical power may be provided to apply an electrical signal to the third electrode pattern 300, independently of the first electrode pattern 100, that is at least two electrical power unit may be provided in the device. When the third electrode pattern 300 and the fourth electrode pattern 400 are provided and driven independently of the first electrode pattern 100 and the second electrode pattern 200, the fringe field generated on the top side of the support (the side provided with the first electrode pattern 100 and the second electrode pattern 200) and the fringe field generated on the bottom side of the support (the side provided with the third electrode pattern 300 and the fourth electrode pattern 400) may have different shapes. This may be particularly useful when the top side and the bottom side of the support have different shapes and sizes, or when the top side and the bottom side of the support are exposed to different environments. Accordingly, in the above case, the third electrode pattern 300 and the fourth electrode pattern 400 may have different patterns from the first electrode pattern 100 and the second electrode pattern 200.

As the fringe field is also generated between the third electrode pattern 300 and the fourth electrode pattern 400, the fringe field may be generated on both the top and bottom sides of the support. In this case, since the biofilm may be grown on both the top and bottom sides of the support and its growth may be regulated, the biofilm growth and control area may be widened. Therefore, the effect of removing organic pollutants by the biofilm may be improved.

Although the drawing illustrates that four electrode patterns are provided, the number of electrode patterns may be further increased, as needed. For example, an electrode structure may also be configured such that a total of eight electrode patterns are provided by providing two interdigitated electrode patterns each on the top, bottom, and both sides of a rectangular parallelepiped support.

In the electrode structure according to one embodiment of the present disclosure, since the fringe field, which is an electric field, is generated on the surface of the electrode structure, it is possible to regulate biofilm growth using the electric field even though the biofilm is not provided between electrodes. This is advantageous in that its applicability is excellent, as compared to an electrode structure in which two opposing electrodes are placed and an electric field generated between the opposing electrodes is used.

The electrode structure described above may be used as a water treatment device. The water treatment device includes the above-described electrode structure and the biofilm provided on the surface of the electrode structure, in which the biofilm may remove organic pollutants in water passing through the water treatment device.

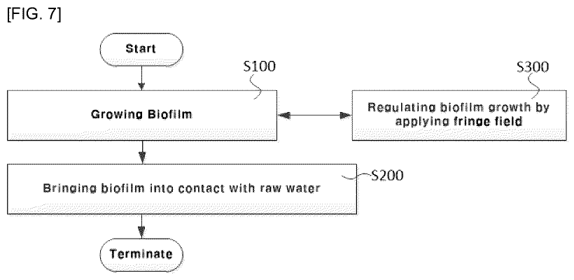

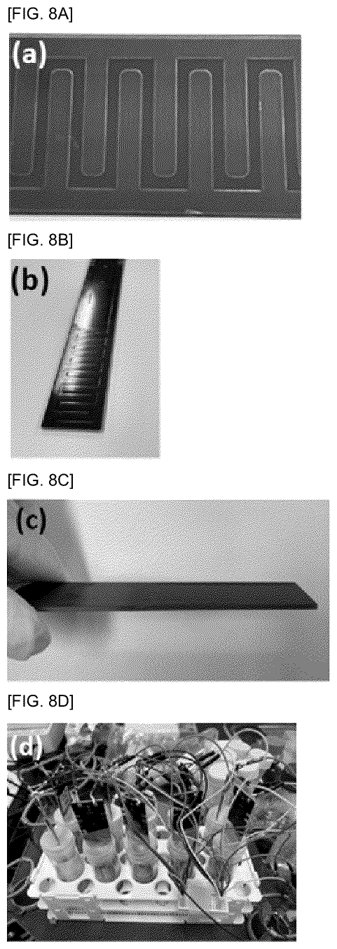

FIG. 4 shows a cross-sectional view of a water treatment device including the electrode structure according to one exemplary embodiment of the present disclosure, and FIG. 5 shows cross-sectional views of water treatment devices according to the prior art.

Referring to FIG. 4, the water treatment device according to one exemplary embodiment of the present disclosure includes the first electrode pattern including the plurality of electrode fingers extending in one direction; the second electrode pattern which is provided between the plurality of electrode fingers provided in the first electrode pattern to form the interdigitated pattern with the first electrode pattern; and the biofilm which is provided on the surface of at least one of the first electrode pattern and the second electrode pattern, wherein the biofilm is provided in the flow path of raw water or in a chamber into which raw water is introduced so that the biofilm may be brought into contact with raw water to be treated, thereby removing organic pollutants in raw water, and a fringe field is generated between the first electrode pattern and the second electrode pattern to regulate growth of the biofilm.

As shown in the drawing, in the water treatment device according to one exemplary embodiment of the present disclosure, the fringe field is generated between the plurality of electrode fingers provided in the electrode structure. Accordingly, the fringe field is generated such that it diverges from the surfaces of the electrode structure and then converges to the surfaces thereof again. Since the fringe field is generated as described above, it is possible to regulate biofilm growth using the electric field, even though the biofilm is provided on the surface of the electrode structure according to one embodiment of the present disclosure.

Compared to this, an electrode structure, in which opposing electrodes are placed, has a problem that regulation of the biofilm growth is possible only when the biofilm exists between the two opposing electrodes.

Referring to FIG. 5, in the traditional case, a straight electric field generated between two opposing electrodes was employed, and bare electrodes exposed to the outside were also employed. However, this type of electrode structure is not suitable for wastewater treatment. Bare electrodes are undesirable because they result in Faradaic currents and generate galvanic byproducts such as metal ions and free radicals. Also, since the area where the electric field is generated is limited to the area between the two electrodes, the electric field may be applied to the biofilm only when the biofilm is provided between the electrodes. In this case, since raw water to be treated flows between the two electrodes provided with the biofilm, there has been a problem that the flow rate of raw water is determined according to the gap between the electrodes. When the gap between the electrodes was increased to increase the flow rate of raw water, a high voltage had to be applied to the electrodes in order to generate an electric field capable of regulating the biofilm growth. However, when a high voltage is applied, galvanic by-products such as metal ions and free radicals may be generated, as described above, which may cause problems in water treatment. In addition, when the gap between the electrodes is narrowed, it may be operated at a relatively low voltage, but as described above, there is a problem that the flow rate of raw water is reduced.

In contrast, the water treatment device according to one exemplary embodiment of the present disclosure allows water to flow freely while the electrode structure operates at a low voltage. In the electrode structure, the fringe field is generated between adjacent electrode fingers (electrode fingers of the first electrode pattern and electrode fingers of the second electrode pattern), and the two electrode fingers are located very close to each other, and accordingly, it is possible to generate the electric field even with low electrical power.

The first electrode pattern and the second electrode pattern of the electrode structure may be covered with an insulating layer. The biofilm grows on the surface of the insulating layer, and growth of the biofilm is regulated by a small oscillating (alternating current, AC)) signal at low frequency. The insulating layer may prevent any ohmic and galvanic interaction with the bacterial culture during the growing of biofilm.

The water treatment device may be further provided with an electrical power controlling unit for generating the fringe field by applying electrical power to the first electrode pattern when the thickness of the biofilm 20 is examined and is greater than a predetermined value. The examination of the thickness of the biofilm 20 may be performed by various methods, such as by reading a ruler installed on the surface of the electrode structure or by using an optical method.

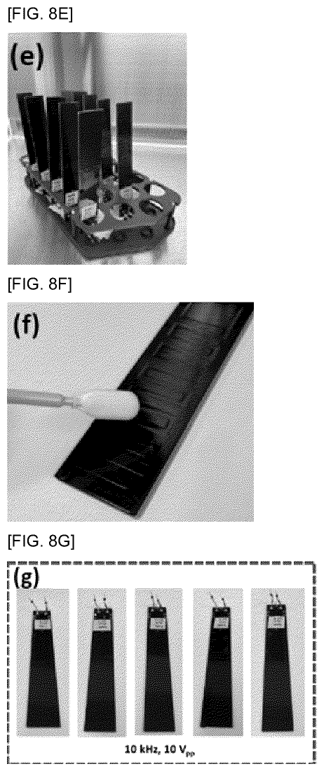

Next, FIG. 6 shows a cross-sectional view of the water treatment device according to one exemplary embodiment of the present disclosure.

Referring to FIG. 6, the plurality of electrode structures 10 may be provided in the water treatment device. The biofilm 20 is provided on the surface of each electrode structure 10. Raw water is introduced along the flow path of the water treatment device, and discharged after removing organic pollutants by the biofilm 20.

At this time, the plurality of electrode structures 10 may be provided along the flow path, and raw water introduced into the water treatment device may be treated by the electrode structures. In some cases, the electrode structures may be provided in a batch type, as shown in FIG. 4, rather than in a type in which water is discharged through a pipe-type flow path after treatment, as disclosed in the drawing.

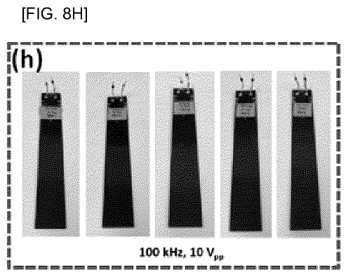

FIG. 7 shows a flowchart illustrating a water treatment method according to one exemplary embodiment of the present disclosure.

Referring to FIG. 7, the water treatment method according to one exemplary embodiment of the present disclosure includes the steps of growing the biofilm on the electrode structure (S100); bringing the electrode structure, to which the biofilm is attached, into contact with raw water to be treated (S200); generating a fringe field from the electrode structure by examining the growth state of the biofilm (S300).

The step of growing the biofilm (S100) may be performed by selecting microorganisms, transplanting the microorganisms to the surface of the electrode structure, and creating an environment in which the microorganisms are able to grow. Alternatively, the step may be performed by growing the microorganisms in the form of a film in another place, and transplanting the same to the surface of the electrode structure.

Next, in the step of bringing the biofilm-attached electrode structure into contact with the raw water (S200), as discussed above, raw water is introduced into the water treatment device in a batch or pipe type, and then brought into contact with the electrode structure to which the biofilm is attached. The contact time between the biofilm and the raw water may vary depending on the condition of raw water. In addition, a step of physically filtering raw water may be additionally performed before bringing raw water into contact with the biofilm, as needed.

Next, the step of generating the fringe field from the electrode structure by examining the growth state of the biofilm (S300) is performed. To generate the fringe field, oscillating electrical power may be applied to the electrode structure, as described above. In addition, the intensity of the fringe field may be determined by considering the growth state and thickness of the biofilm, etc.

Hereinabove, the electrode structure, the water treatment device, and the water treatment method according to one exemplary embodiment of the present disclosure have been described. Hereinafter, the electrode structure and the water treatment device will be described with reference to Experimental Examples.

Hereinafter, the electric field strength passing through the biofilm at varying distances from the surface was estimated via a two-dimensional electric field simulation, which allows to also examine the effects of biofilm on the electric field distribution. The electrode structure of the fringe field design was fabricated using printed circuit boards and used as surfaces for biofilm attachment and growth.

### Preparation Example 1. Design of Electrode Structure

The proposed fringe field design electrode structure consisted of electrode fingers interlacing each other, or interdigitated electrodes. The interdigitated electrode is a design to maximize the overlapping areas between two electrodes.

The electrodes may also be fabricated using two-dimensional stenciling techniques (e.g., ink-jet printing or screen printing) on rigid or flexible supports.

The electrode fingers are supported on both the top side and bottom side of a fiberglass support. The electrode fingers are also coated with a thin layer of epoxy insulation (thickness of about 20 μm) to prevent any ohmic and galvanic interaction with the bacterial culture during the growing of biofilm. The key dimensions of the electrode structure are summarized in Table 1.

TABLE 1

| Features | Dimensions | |
|---|---|---|
| Total Length | 155 | mm |
| Total Width | 24 | mm |
| Total Thickness | 1.6 | mm |
| Electrode Finger Thickness | 0.06 | mm |
| Electrode Finger Length | 15 | mm |
| Electrode Finger Width | 5 | mm |
| Electrode Finger Spacing | 1.5 | mm |
| Number of Electrode Fingers per Polarity | 7 | |

In order to facilitate the generation of a fringe field between the electrode fingers, solid core wires (diameter of about 0.64 mm) were soldered to the electrical connections. This would allow oscillating electrical signals to be sent to an electrode pattern set while the opposing electrode pattern is grounded. In this way, an oscillating fringe field may be generated between the two electrode patterns, and it would pass through any biofilm on the electrode pattern. The same wiring configuration was used for both the top and bottom sides of the support.

### Preparation Example 2. Modeling of Fringe Field by Electrode Structure

The electric field distribution at the surface of the fringe field electrode structure was modeled. Since the target electric field is that directly above an electrode finger in the y-direction (E q), a two-dimensional model would suffice. The model consisted of five electrode fingers at the base of a 10 mm×34 mm dielectric block (designated as water with relative electrical permittivity εᵣ=81). Since a 10 Vₚₚ (peak to peak) sinusoidal wave was used as the oscillating electrical signal, the electrical potentials were assigned to the electrode fingers (left to right) as 10 V, 0 V, 10 V, 0 V, 10 V to simulate the maximum electric field during operation. After computing the finite element model, the E_{y} field strength (V/m) versus distance (mm) from the electrode finger surface was plotted.

The difference in the E_{y} field strength with and without biofilm was also examined using a simplified version of the above-mentioned model. The number of electrode fingers was reduced from five to three to facilitate efficient computation. Two dielectric blocks were employed and assigned as water (thickness of 3.5 mm, relative electrical permittivity εᵣ of 81) and biofilm (thickness of 0.5 mm, relative electrical permittivity εᵣ=40). The E_{y} field strength without biofilm was computed by assigning both dielectric blocks as water. The E_{y} field strength with biofilm was computed by assigning the lower dielectric block as biofilm. After computation, the E_{y} field strength (V/m) versus distance (mm) from the electrode finger surface was plotted for both scenarios.

### Preparation Example 3. Formation of Biofilm on Surface of Electrode Structure

In detail, Escherichia coli K12 was chosen as a model bacterium. E. coli K12 was incubated into a 250 mL flask containing 100 mL of Luria-Bertani broth (BD Biosciences, San Jose, USA). The culture was incubated at ambient temperature for 24 hours with a shaking speed of 100 rpm. The optical density was analyzed by measuring the absorbance at 600 nm (OD 600 nm) using a spectrophotometer. The 15 mL culture incubated at ambient temperature for 24 hours was transferred to 100 mL of clean LB broth. The growth curve of E. coli K12 bacterial culture was analyzed by measuring optical density (OD 600 nm) using the spectrophotometer as described for 48 hours.

The electrode structure was dipped inside a 50 mL conical tube with 30 mL of an E. coli K12 bacterial culture. The electrode fingers were submerged within the bacterial culture, and the top of the conical tube was sealed to minimize evaporation. The electrode structure was electrically connected to a function generator via the soldered solid-core wires. The fringe field was generated by driving the electrodes with AC signal at frequencies of 10 kHz and 100 kHz (sinusoidal wave) with 10 Vₚₚ (peak to peak voltage). There were five fringe field electrode structures operating at each frequency (a total of 10 devices for both frequencies). Two additional fringe field electrode structures were designated as a control (frequency of 0 kHz at 0 Vₚₚ). All twelve electrode structures were simultaneously in the bacterial culture (one device per conical tube). The submersion of the fringe field electrode structures lasted for approximately 4 days at ambient temperature.

Next, after removing the fringe field electrode structure from the conical tube, it was dip-rinsed gently three times into a beaker of de-ionized water in order to wash off any bacteria that were not part of the biofilm. After rinsing, the fringe field electrode structures were air-dried vertically in a hood. Once the fringe field electrode structures were dried, swabs moistened with de-ionized water were used to harvest the biofilm.

The swab that harvested the biofilm was separated from its handle and immersed in 3 mL of de-ionized water in 5 mL tube, followed by sonication in a water bath for 1 minute to loosen the harvested biofilm from the swab. After sonication, the biofilm was subjected to ATP and optical density measurement.

### Experimental Example 1. Fabrication of Electrode Structure and Electrical Power Application Test

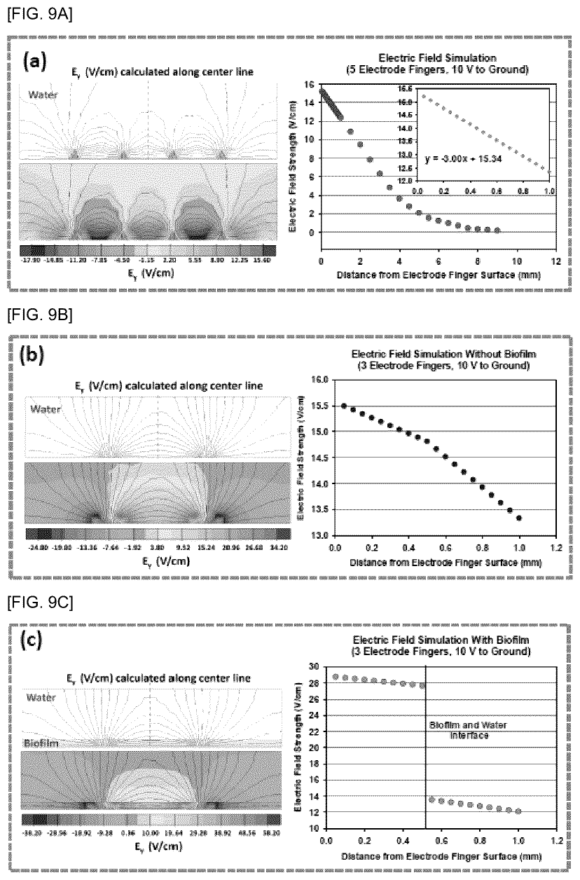

FIG. 8A to 8H show a method of preparing the electrode structure according to one exemplary embodiment of the present disclosure.

The interdigitated electrodes on the electrode structure were spaced by a well-dimensioned gap. The corners of the electrode fingers were slightly rounded off as a result of the manufacturing process (FIG. 8A). As shown in FIG. 8D, due to the large number of electrode structures running at the same time, the wirings over the slow shaker had to cross over each other. However, this was not expected to cause cross-talk or interference due the use of low frequencies (100 kHz or less). The air-drying process after dip-rinsing resulted in a coffee ring effect away from the electrode fingers instead of on them (FIG. 8E). The biofilm harvesting process was probably the most vulnerable to variability (FIG. 8F). It was performed by the same individual using the same swabbing motion for all of the electrode structures. As shown in FIGS. 8G and 8H, as expected, the air-dried biofilm was barely visible to the naked eye.

### Experimental Example 2. Generation and Analysis of Fringe Field

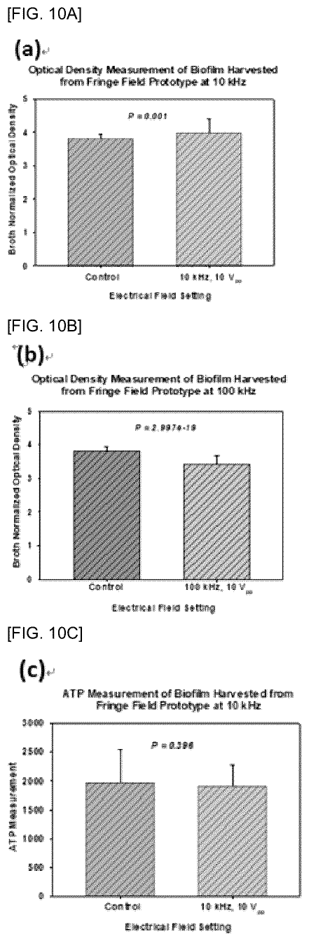

FIG. 9A to 9C show results of analyzing the fringe field generated by the electrode structure.

The fringe field distribution over five electrode fingers is shown in FIG. 9A. The maximum field strength achieved was 15.19 V/cm (distance from electrode surface=0.05 mm). This field strength decreased linearly as the distance from electrode surface increased from 0.05 mm to 1 mm (y=−3.00×+15.3). At 1 mm, the field strength was 12.3 V/cm. Since the biofilm thickness on the electrode surface is definitely less than 1 mm, this means that the biofilm was subjected to an electric field of at least 12.3 V/cm. This is approximately 2 V/cm to 4 V/cm higher than observed at the bare electrode. In general, biofilm thickness does not exceed 2 mm. At 2 mm, the electric field strength of the fringe field device was still higher than that due to the bare electrode at 9.43 V/cm.

After modification to allow the inclusion of a biofilm layer in the simulation model, there was a slight change in the simulation results (FIG. 9B). The electric field strength at 0.05 mm increased from 15.2 V/cm to 15.6 V/cm. Similarly, the electric field strength at 1 mm increased from 12.3 V/cm to 13.3 V/cm. Nonetheless, it did not impact the conclusion drawn from this simulation. As shown in FIG. 9C, the presence of the biofilm (thickness of about 0.5 mm) induced electric field lines to concentrate within it. This is because the biofilm has a lower relative permittivity (εᵣ=40) than water (εᵣ=81). As a result, the electric field strength within the biofilm almost doubled to 28.75 V/cm at 0.05 mm away from the electrode surface. The discontinuity of the electric field distribution became distinct at the biofilm and water interface. The significance of this simulation result means that as biofilm grows on a fringe field device, the permeated electric field gets stronger and growth inhibition becomes more pronounced.

### Experimental Example 3. Measurement of Optical Density of Biofilm

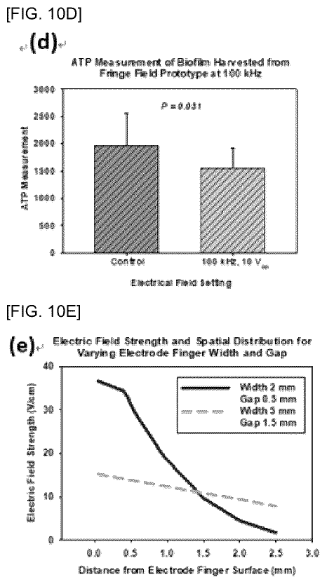

FIG. 10A to 10E are graphs showing results of measuring optical density of a biofilm fabricated according to one exemplary embodiment of the present disclosure.

The optical density (OD) of each biofilm harvest is normalized with the optical density (OD) of the broth in which the fringe field electrode structure was submerged as follows:

Broth Normalized OD=(Biofilm Harvest OD)/(Broth OD)×100 (1)

As shown in FIGS. 10A and 10B, the normalized optical densities for control, 10 kHz, and 100 kHz were 3.81±012, 4.00±0.40, and 3.43±0.26, respectively. At 10 kHz, the normalized optical density turned out to be higher than that of the control. However, as expected, that at 100 kHz was significantly lower than that of the control. This means that when the fringe field electrode structure was driven at 100 kHz, the reduction in normalized optical density was approximately 10.06%. This means that the fringe field of the electrode structure of the present disclosure is definitely able to regulate bacterial biofilm growth on its surface.

The extent of reduction due to the fringe field approach and its dependence on frequency became more evident with the ATP measurements, as they are indicative of the bacterial cell viability. As shown in FIG. 10C, the ATP measurements suggested that there is no significant difference in terms of bacterial cell viability between the control (1,975±577) and at 10 kHz (1,918±368). However, at the driving frequency of 100 kHz, the ATP measurement was reduced to 1568±355. Therefore, the fringe field driven at 100 kHz was able to reduce the bacterial viability in the biofilm by one-fifth.

One of the key advantages of using the fringe field is the ability to increase the electric field density near the surface by simply reducing the electrode finger width or the gap between the electrode fingers without changing other parameters (voltage, etc.). In other words, it is possible to adjust the density and extent of the fringe field in the bioreactor without having to change its fundamental design. As shown in FIG. 10E, by changing the electrode finger's width from 5 mm to 2 mm and the gap from 1.5 mm to 0.5 mm, the electric field strength at 0.05 mm from the surface increased from 15.2 V/cm to 36.6 V/cm. However, at 2 mm from the surface, the electric field strength has become much lower at 4.48 V/cm.

Another key advantage of using the fringe field is that it is highly scalable because it can be adapted for different surface contours while maintaining its field strength. The support does not need to be restricted to a flat surface. For example, the electrode fingers may be made to conform over curved topologies within the bioreactor. The electrode fingers may be easily screen-printed on flexible materials such as polyamide and polyester.

In the present disclosure, the reduction of bacterial biofilm growth using fringe field generated by an interdigitated electrode pattern was confirmed. The electric field simulations (using a safe voltage of 10 V) suggested a maximum field strength of 15.2 V/cm at 0.05 mm away from the surface. At a distance of 1 mm from the electrode surface, the field strength was 12.3 V/cm and sufficient to disrupt biofilm growth. With the inclusion of biofilm in the simulation, the fringe field became denser near the surface (from 15.6 V/cm to 28.8 V/cm at 0.05 mm) due to the difference in relative permittivity. This indicates a benefit that the biofilm growth may be reduced at the same voltage. The optical density for biofilm harvest at 100 kHz (3.43±0.26) was significantly lower than that of the control (3.81±012). In terms bacterial cell viability, the efficacy of the fringe field at 100 kHz became more apparent where the ATP measurement was reduced by 20.61% from 1,974±577 to 1,567±355. By narrowing the width and gap of the electrode fingers, the fringe field may be further intensified multiple times. This suggests that the electric fringe field may be readily employed to regulate biofilm growth in the bioreactor for wastewater treatment.

Hereinabove, although the present disclosure has been explained with reference to preferred exemplary embodiments thereof, it will be understood by a person skilled in the art or a person having ordinary knowledge in the art that the present disclosure may be variously modified and changed without departing from the spirit and technical scope of the present disclosure described in the claims to be described later.

Accordingly, the technical scope of the present disclosure should be defined by the claims rather than the detailed description of the specification.

```
[Reference numerals]
  10: Electrode structure
  20: Biofilm
100: First electrode pattern
110: Electrode finger
200: Second electrode pattern
210: Electrode finger
500: Support
```

### EFFECT OF THE INVENTION

According to one embodiment of the present disclosure, biofilm growth may be inhibited by using a fringe field generated on the surface of an electrode structure, and the fringe field is not generated between opposing electrodes, but generated in a divergent and convergent form on the surface of the electrode structure, and thus it is possible to inhibit biofilm growth while ensuring high water throughput.

What is claimed is:

1. An electrode structure comprising:
a first electrode pattern including a plurality of electrode fingers extending in one direction; and
a second electrode pattern which is provided between the plurality of electrode fingers provided in the first electrode pattern to form an interdigitated pattern with the first electrode pattern,
wherein both the first electrode pattern and the second electrode pattern are covered with a electrical insulating layer,
wherein a fringe field is generated between the first electrode pattern and the second electrode pattern to regulate growth of a biofilm which is provided on the surface of at least one of the first electrode pattern and the second electrode pattern.

2. The electrode structure of claim 1, wherein the first electrode pattern is connected with electrical power, and the second electrode pattern is grounded.

3. The electrode structure of claim 2, wherein oscillating electrical signals are applied to the first electrode pattern.

4. The electrode structure of claim 1, wherein the first electrode pattern and the second electrode pattern are provided on the same side of a support.

5. The electrode structure of claim 4, further comprising a third electrode pattern and a fourth electrode pattern which are provided on the side other than the side of the support on which the first electrode pattern and the second electrode pattern are provided,
wherein the third electrode pattern includes a plurality of electrode fingers extending in one direction, and
the fourth electrode pattern is provided between the plurality of electrode fingers provided in the third electrode pattern to form an interdigitated pattern with the third electrode pattern,
wherein a fringe field is generated between the third electrode pattern and the fourth electrode pattern.

6. The electrode structure of claim 5, wherein the first electrode pattern is connected with the third electrode pattern and the second electrode pattern is connected with the fourth electrode pattern.

7. The electrode structure of claim 5, wherein the first electrode pattern is electrically separated from the third electrode pattern and the second electrode pattern is electrically separated the fourth electrode pattern,
and wherein each of the first electrode pattern and the third electrode pattern is connected with different electrical power.

8. The electrode structure of claim 1, wherein the first electrode pattern and/or the second electrode pattern are formed of a metal, graphene or indium tin oxide (ITO).

9. The electrode structure of claim 1, wherein the first electrode pattern and the second electrode pattern are interdigitated with each other to form an acute or obtuse angle.

10. A water treatment device comprising:
a first electrode pattern including a plurality of electrode fingers extending in one direction;
a second electrode pattern which is provided between the plurality of electrode fingers provided in the first electrode pattern to form an interdigitated pattern with the first electrode pattern; and
wherein both the first electrode pattern and the second electrode pattern are covered with a electrical insulating layer,
a biofilm which is provided on the surface of at least one of the first electrode pattern and the second electrode pattern,
wherein the biofilm is provided in the flow path of raw water or in a chamber into which raw water is introduced so that the biofilm is brought into contact with raw water to be treated, thereby removing organic pollutants in raw water, and
a fringe field is generated between the first electrode pattern and the second electrode pattern to regulate growth of the biofilm.

11. The water treatment device of claim 10, wherein the biofilm is provided on the insulating layer.

12. The water treatment device of claim 10, further comprising an electrical power controlling unit for generating a fringe field by applying electrical power to the first electrode pattern when the thickness of the biofilm is examined and is greater than a predetermined value.

13. The water treatment device of claim 10, further comprising a third electrode pattern and a fourth electrode pattern which are provided on the side other than the side of the support on which the first electrode pattern and the second electrode pattern are provided,
wherein the third electrode pattern includes a plurality of electrode fingers extending in one direction, and
the fourth electrode pattern is provided between the plurality of electrode fingers provided in the third electrode pattern to form an interdigitated pattern with the third electrode pattern,
wherein a fringe field is generated between the third electrode pattern and the fourth electrode pattern.

14. The water treatment device of claim 13, wherein at least two electrical power unit is provided.

15. The water treatment device of claim 10, wherein the distance between the electrode fingers provided in the first electrode pattern and the electrode fingers provided in the second electrode pattern is adjusted according to the thickness of the biofilm and the throughput of the raw water.

16. A water treatment method, the method comprising the steps of:
growing a biofilm on an electrode structure;
bringing the electrode structure, to which the biofilm is attached, into contact with raw water to be treated; and
generating a fringe field from the electrode structure by examining the growth state of the biofilm.

17. The water treatment method of claim 15, wherein the electrode structure includes a first electrode pattern including a plurality of electrode fingers extending in one direction; and a second electrode pattern which is provided between the plurality of electrode fingers provided in the first electrode pattern to form an interdigitated pattern with the first electrode pattern; and wherein the fringe field is generated between the first electrode pattern and the second electrode pattern to regulate growth of the biofilm which is provided on the surface of at least one of the first electrode pattern and the second electrode pattern.

18. The water treatment method of claim 15, wherein the electrode structure further comprising a third electrode pattern and a fourth electrode pattern which are provided on the side other than the side of the support on which the first electrode pattern and the second electrode pattern are provided,
wherein the third electrode pattern includes a plurality of electrode fingers extending in one direction, and
the fourth electrode pattern is provided between the plurality of electrode fingers provided in the third electrode pattern to form an interdigitated pattern with the third electrode pattern,
wherein a fringe field is generated between the third electrode pattern and the fourth electrode pattern.

19. The water treatment method of claim 18, wherein the first electrode pattern is electrically separated from the third electrode pattern and the second electrode pattern is electrically separated the fourth electrode pattern,
wherein each of the first electrode pattern and the third electrode pattern is connected with different electrical power,
and wherein the third electrode pattern and the fourth electrode pattern are driven independently of the first electrode pattern and the second electrode pattern.

20. The water treatment method of claim 18, wherein the biofilm as lower relative permittivity than water.
